(19) United States
(12) Patent Application Publication
(10) Pub. No.: US 2024/0416006 A1
(43) Pub. Date: Dec. 19, 2024

(54) FISTULA FILLER AND DEPLOYMENT SYSTEM

(71) Applicant: Miromatrix Medical Inc., Eden Prairie, MN (US)

(72) Inventors: Dominique Davidow, Minnetonka, MN (US); Aleksandr Katane, Victoria, MN (US); Ben Steiner, Bloomington, MN (US)

(21) Appl. No.: 18/817,498

(22) Filed: Aug. 28, 2024

Related U.S. Application Data
(63) Continuation of application No. 17/251,573, filed on Dec. 11, 2020.
(60) Provisional application No. 62/684,426, filed on Jun. 13, 2018.

(51) Int. Cl. A61L 27/36; A61L 27/04; A61L 27/14

(52) U.S. Cl. CPC A61L 27/3633; A61L 27/04; A61L 27/14; A61L 27/3641; A61L 27/3683; A61L 2430/40

(57) ABSTRACT

Disclosed herein are methods to maintain or inhibit an alteration in volume or shape of hydrated decellularized extracellular matrix from a mammalian organ or tissue or a portion thereof.

### CROSS REFERENCE

This application is a continuation of U.S. patent application Ser. No. 17/251,573, titled “FISTUAL FILLER AND DEPLOYMENT SYSTEM” and filed Dec. 11, 2020, which is a 371 national phase application of PCT Application Serial No. PCT/US2019/036942, titled “FISTUAL FILLER AND DEPLOYMENT SYSTEM” and filed Jun. 13, 2019, which claims the benefit of priority to U.S. Provisional Patent Application Ser. No. 62/684,426, titled “FISTUAL FILLER AND DEPLOYMENT SYSTEM” and filed Jun. 13, 2018, each of which is incorporated by reference herein in its entirety . . .

### BACKGROUND

A fistula is an abnormal anastomosis, that is, an abnormal connection between a set of organs or vessels, e.g., where there are two hollow spaces (technically, two epithelialized surfaces), such as blood vessels, intestines, or other hollow organs. The connection may take on the form of a tract which is often difficult to heal or close. Fistulas can be caused by injury or surgery, but they can also result from an infection or inflammation. Fistulas are generally a disease condition, but they may be surgically created for therapeutic reasons.

Surgery is often required to assure adequate drainage of the fistula (so that pus may escape without forming an abscess). Various surgical procedures can be commonly used, most commonly fistulotomy, placement of a seton (a cord that is passed through the path of the fistula to keep it open for draining), or an endorectal flap procedure (where healthy tissue can be pulled over the internal side of the fistula to keep feces or other material from reinfecting the channel). Current treatment often involves filling the fistula with fibrin glue or plugging it with plugs made of porcine small intestine submucosa. Surgery for fistulae, e.g., anorectal fistulae, is not without side effects, including recurrence, reinfection and incontinence.

### SUMMARY

The retention of the shape of decellularized tissue or of a portion of an organ can be accomplished through the irradiation of the decellularized tissue or the portion of the organ inside a shaping mold. The enclosure of decellularized tissue or a portion of an organ inside of a mold or other constraining material, such as stainless steel or platinum or polymers such as polytetrafluorocthylene (PTFE) or polycaprolactone (PCL), allows the tissue to take on the shape of the mold or constraint and subsequently retain that shape after it is irradiated. This can result in decellularized extracellular matrix having defined (pre-determined) shapes, e.g., for therapeutic uses. For example, the defined shape may be introduced to a deployment system such as one for surgical placement of a fistula filler, e.g., a product that takes on the shape of the fistula, or a plug, e.g., a product that displaces the area of the fistula in order to fit inside the fistula and can be in one embodiment more rigid than a fistula filler. The system can include a hollow device which contains the filler or plug. The system may be inserted into the fistula tract and the filler or plug may be deployed by pulling, pushing or otherwise expelling the filler or plug into the tract. The deployment device can then be removed from the tract leaving behind the filler or plug within the fistula tract.

Thus, disclosed herein are methods to maintain or inhibit an alteration in volume or shape of hydrated decellularized extracellular matrix from a mammalian organ or tissue or a portion thereof. A “hydrated decellularized extracellular matrix” can be a decellularized extracellular matrix that is not fully dry, e.g., it is at least partially hydrated with an aqueous solution such as deionized water, or saline, e.g., phosphate buffered saline. The method can include at least partially reducing malleability of an isolated at least partially hydrated at least partially decellularized organ or portion thereof in a mold. In some embodiments, a method can comprise at least partially reducing malleability. In some cases, a method can comprise exposing an at least partially hydrated at least partially decellularized organ or portion thereof to a select dose of radiation. In some embodiments a method can include providing a hydrated decellularized extracellular matrix from a mammalian organ or tissue or a portion thereof; placing or introducing the hydrated decellularized extracellular matrix from the mammalian organ or tissue or the portion thereof into a mold having a predefined shape which optionally can be in packaging, e.g., a pouch, tray, vial, or sterile container; and subjecting the hydrated decellularized extracellular matrix from the mammalian organ or tissue or the portion thereof in the mold to a selected dose of radiation to provide for maintenance or inhibition of an alteration in the volume or shape of the hydrated decellularized extracellular matrix that is in the mold. In another embodiment, a hydrated decellularized extracellular matrix from a mammalian organ or tissue or a portion thereof can be placed or introduced into a mold having a predefined shape which optionally can be in packaging, e.g., a pouch, tray, vial, or sterile container; subjecting the hydrated decellularized extracellular matrix from the mammalian organ or tissue or the portion thereof in the mold to dehydration; and subjecting the dehydrated decellularized extracellular matrix in the mold to a selected dose of radiation, e.g., to provide for maintenance or inhibition of an alteration in the volume or shape of the decellularized extracellular matrix that is in the mold. In one embodiment, the decellularized extracellular matrix from the mammalian organ or tissue or a portion thereof can be hydrated in saline, e.g., 0.9% normal saline or phosphate buffered saline, water or autologous serum, plasma, e.g., platelet rich plasma. In one embodiment, the decellularized extracellular matrix from the mammalian organ or tissue or a portion thereof can be placed into a preselected mold. In one embodiment, the hydrated decellularized extracellular matrix from the mammalian organ or tissue or portion thereof can be pressed into the mold. In one embodiment, multiple layers of hydrated decellularized extracellular matrix from the mammalian organ or tissue or a portion thereof can be placed or pressed into the mold. In one embodiment, the mammal can be a pig or human. In one embodiment, the organ can be a liver, muscle, lung, spleen or heart. In one embodiment, the dose of radiation can be about 5 kGy to 50 kGy. In one embodiment, the dose of radiation can be about 5 kGy to 15 kGy. In one embodiment, the dose of radiation can be about 15 kGy to 25 kGy. In one embodiment, the dose of radiation can be about 25 kGy to 50 kGy. In one embodiment, the radiation can be E-beam radiation or gamma radiation. In one embodiment, the radiation can be thermal or UV radiation. In one embodiment, after irradiation, the decellularized extracellular matrix can be dried. In one embodiment, the hydrated portion can be about 0.25 cm×0.25 cm, e.g., 1 cm×1 cm, to about 60 cm×60 cm (length×width). In one embodiment, the hydrated portion can be about 0.5 cm×0.5 cm×0.5 cm to about 30 cm×30 cm×30 cm. In one embodiment, the hydrated portion thereof can be about 1 cm×1 cm (length×width) to about 10 cm×10 cm. In one embodiment, the hydrated portion can be about 1 cm×2 cm to about 10 cm×12 cm. In one embodiment, the hydrated portion can be about 1 cm×6 cm to about 3 cm×20 cm. In one embodiment, the hydrated portion for anal uses can be about 0.5 cm×4 cm to about 3×10 cm, for vaginal uses can be about 0.5 cm×4 cm to about 3 cm×10 cm, for biliary uses can be about 0.2 cm×2 cm to about 3 cm×1 cm, for gastroenterological use can be about 0.2 cm×2 cm to about 3×10 cm, for bladder uses can be about 0.5 cm×4 cm to about 3 cm×10 cm, for esophageal uses can be about 0.2 cm×2 cm to 3 cm×10 cm, for nerve wraps can be about 0.2 cm (diameter)×1 cm length to about 2 cm×10 cm, or for tendon wraps can be about 1 cm×2 cm to about 4 cm×12 cm. In one embodiment, the portion can be obtained from a decellularized mammalian organ or tissue. In one embodiment, the portion can be obtained from the mammalian organ or tissue before decellularization. In one embodiment, the hydrated decellularized extracellular matrix from the mammalian organ or tissue or the portion thereof prior to radiation, can be inflated with a gas or vapor. In one embodiment, the hydrated portion, prior to and after radiation, has the shape of a cube, rectangular prism or an irregular strip. In one embodiment, the cube can be about 1 cm×1 cm to about 10 cm×10 cm. In one embodiment, the rectangular prism can be about 1 cm×2 cm to about 10 cm to about 12 cm. In one embodiment, an irregular strip about 1 cm×6 cm to about 3 cm to about 20 cm. In one embodiment, the molded and irradiated portion can be about 1 cm×1 cm to about 60 cm×60 cm. In one embodiment, the molded and irradiated portion can be about 0.5 cm×0.5 cm×0.5 cm to about 30 cm×30 cm×30 cm. In one embodiment, the molded and irradiated portion thereof can be about 1 cm×1 cm to about 10 cm×10 cm. In one embodiment, the molded and irradiated portion can be about 1 cm×2 cm to about 10 cm×12 cm. In one embodiment, the molded and irradiated portion can be about 1 cm×6 cm to about 3 cm×20 cm. In one embodiment, the molded and irradiated decellularized extracellular matrix can be subjected to dehydration.

Also disclosed herein is an isolated irradiated portion of a decellularized extracellular matrix from a mammalian organ or tissue having a defined shape useful for therapy. In one embodiment, the shape of the isolated irradiated portion of a decellularized extracellular matrix can be useful as a nerve or tendon wrap. In one embodiment, the shape can be useful for, for example, anal, vaginal, biliary, gastrointestinal, bladder or esophageal fistula repair. For example, a filler for vaginal repair may be about 0.5 cm×about 4 cm to about 3 cm×about 10 cm; for biliary repair may be about 0.2 cm×about 2 cm to 3 cm×about 10 cm; for gastrointestinal repair may about 0.2 cm×about 2 cm to about 3×about 10 cm; for bladder repair may be about 0.5 cm×about 4 cm to about 3 cm×about 10 cm; or for esophageal repair may be about 0.2 cm×about 2 cm to about 3×about 10 cm. In one embodiment, a nerve wrap may have a diameter of about 0.2 cm and a length of about 1 cm or a diameter of about 2 cm and a length of about 10 cm, and a tendon wrap may have a diameter of about 1 cm and a length of about 2 cm or a diameter of about 4 cm and a length of about 12 cm.

Also disclosed herein are methods of preparing a surgical filler deployment device. The method can include providing a deployment device for a surgical filler; providing the isolated irradiated portion described herein; and introducing the isolated irradiated portion into a hollow portion of the device. In one embodiment, the deployment device can be formed of plastic, metal, a composite, a biologic material, or combination thereof. In one embodiment, the deployment device can be a cylindrical tube, a triangular tube, a square tube, or a rectangular tube. In one embodiment, the deployment device can be at least partially biodegradable. In one embodiment, the deployment device can further comprise a pusher or a plunger.

Uses for the deployment device can include a method of treating a mammal in need of a surgical filler, e.g., where the mammal may be in need of anal, vaginal, biliary, gastrointestinal, bladder or esophageal fistula repair, or abnormal connections caused by trauma wounds. Uses for the isolated irradiated portion of a decellularized extracellular matrix from a mammalian organ or tissue, e.g., without a deployment device, can include use as a nerve or tendon wrap.

Also disclosed herein are kits that can comprise an at least partially hydrated at least partially decellularized extracellular matrix and a mold. In some embodiments a kit can comprise an isolated molded, at least partially irradiated portion of the at least partially decellularized extracellular matrix and a surgical filler deployment device. In some embodiments a kit can comprise an isolated molded, at least partially irradiated portion of an at least partially decellularized extracellular matrix in a sealed container. In some embodiments a kit can be in a cleanroom. In some embodiments a kit can be in a factory.

Also disclosed herein is a system that can comprise an isolated molded, at least partially irradiated portion of an at least partially decellularized extracellular matrix, an input attached to an at least partially irradiated at least partially hydrated at least partially decellularized extracellular matrix, an output attached to an at least partially irradiated at least partially hydrated at least partially decellularized extracellular matrix, growth media, and at least one of: a temperature control apparatus, an atmosphere controlling apparatus, or a humidity controlling apparatus. In some embodiments an at least partially hydrated at least partially decellularized extracellular matrix can be cannulated, and an input and and an output can be attached to an at least partially irradiated at least partially hydrated at least partially decellularized extracellular matrix by a cannula. In some embodiments a system can be in a cleanroom. In some embodiments a system can be in a factory.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which the principles of exemplary embodiments are utilized, and the accompanying drawings of which:

FIG. 1 depicts an image of irradiated decellularized tissue inside of an exemplary shaping mold.

FIG. 2 depicts an image of removal of irradiated decellularized tissue from a shaping mold.

FIG. 3 depicts an image of molded decellularized tissue (it retains the shape of the mold it was irradiated in).

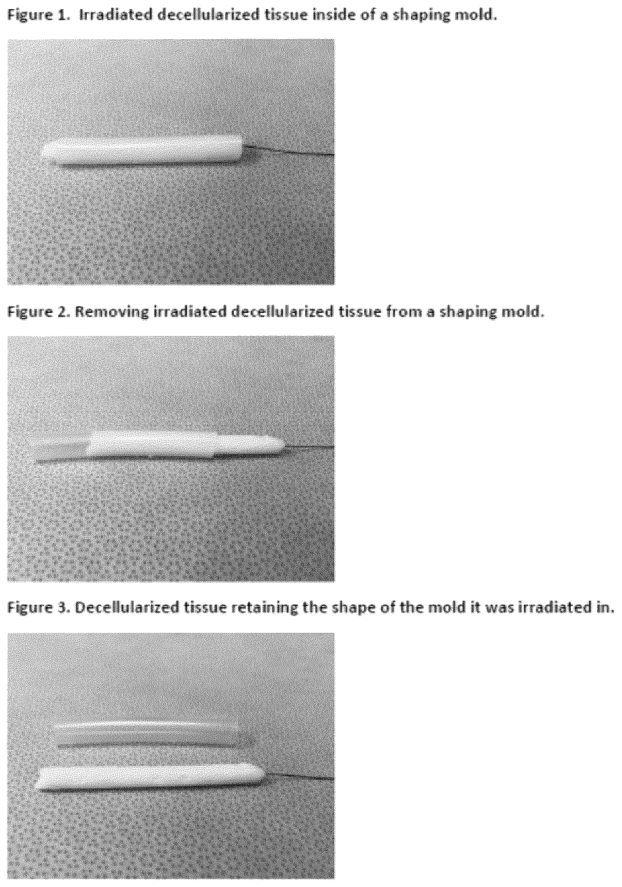

### DETAILED DESCRIPTION

Decellularized tissue from organs or tissues such as liver, lung, muscle, spleen or heart can be cut or pressed into various shapes. However, the decellularized tissue may be limited in the retention of that shape due to the nature of collagen fibers and the empty cellular compartments in the extracellular matrix. To create a decellularized tissue or portion thereof, or a portion of a decellularized organ, that retains a desired shape as described herein, the tissue or portion can be placed into a shaped mold or other constraining device and irradiated. Following irradiation, the decellularized tissue or portion, once removed from the shaping mold or constraining device, retains the shape of the mold or device that the tissue or portion was irradiated in. Thus, this disclosure provides for irradiating decellularized portions of organ or tissues placed into a shaping mold or constraining device to impart a desired shape on the decellularized portion of an organ or tissue. The shape retaining decellularized portion of an organ or tissue can be put to use in a number of applications including but not limited to the use of portions of whole organ or tissue portions thereof as surgical fillers, surgical mesh, wraps, surgical protectors, drug delivery units, surgical connectors or fillers for fistulas and other voids. Alternatively, the shape retaining decellularized portion of an organ or tissue may be seeded with any type of cells following sterilization.

In one embodiment, the irradiation of decellularized tissue or a portion of an organ inside a selected mold increases the tissue or portion memory/recoil. In some embodiments, a dose of radiation can be from about 1 kGy to about 100 kGy, from about 5 kGy to about 100 kGy, from about 10 kGy to about 100 kGy, from about 15 kGy to about 100 kGy, from about 20 kGy to about 100 kGy, from about 25 kGy to about 100 kGy, from about 30 kGy to about 100 kGy, from about 35 kGy to about 100 kGy, from about 40 kGy to about 100 kGy, from about 45 kGy to about 100 kGy, from about 50 kGy to about 100 kGy, from about 55 kGy to about 100 kGy, from about 60 kGy to about 100 kGy, from about 65 kGy to about 100 kGy, from about 70 kGy to about 100 kGy, from about 75 kGy to about 100 kGy, from about 80 kGy to about 100 kGy, from about 85 kGy to about 100 kGy, from about 90 kGy to about 100 kGy, or from about 95 kGy to about 100 kGy.

In one embodiment, the increased shape retention of the decellularized tissue or portion of an organ by irradiating the decellularized tissue or portion of an organ, allows for case of cutting (e.g., trimming), thereby providing for desired three dimensional portions of the matrix. In one embodiment, the molded, irradiated decellularized tissue or portion of an organ can be for use in treatment of fistulas. In one embodiment, the molded, irradiated decellularized tissue or portion of an organ can be for use as a shape retaining surgical filler. In one embodiment, the molded, irradiated decellularized tissue or portion of an organ can be for use as a shape retaining surgical wrap, connector or protector. In one embodiment, the molded, irradiated decellularized tissue or portion of an organ can be for use as a shape retaining surgical wrap. In one embodiment, prior to molding, the decellularized tissue or portion of an organ can be inflated with a gas or vapor, e.g., while inside a mold for a shape retaining surgical filler or for a shape retaining wound matrix.

Disclosed herein are systems which can be composed of a hollow deployment device containing a fistula filler or plug that may be inserted into the fistula tract. The deployment device allows for placement of a soft fistula filler or plug into a fistula tract. The deployment device also allows for easier placement of a long filler or plug into a fistula tract. Placement of a fistula filler or plug can be aided by the hollow device which allows the filler or plug to be placed within the tract prior to removal of the hollow device. The deployment device may be formed of a polymer including but not limited to polytetrafluororethylene, polypropylene, polyethylene, polystyrene, nylon, polyetheretherketone, or polyurethane. In one embodiment, the hollow deployment device may be composed of plastic, metal, composite, biologic material, or combination thereof. In one embodiment, the hollow deployment device may be a cylindrical tube, triangular tube, square tube, rectangular tube or some combination thereof. In one embodiment, the hollow deployment device may have non-rigid walls to facilitate placement of the molded, irradiated decellularized tissue or portion of an organ in a non-linear tract. In one embodiment, the hollow deployment device may have non-rigid walls to facilitate expelling of the molded, irradiated decellularized tissue or portion of an organ into the tract. In one embodiment, the hollow deployment device may have an opening in the wall such as a slit or hole to facilitate initial placement of the molded, irradiated decellularized tissue or portion of an organ into the hollow device during manufacturing. In one embodiment, the molded, irradiated decellularized tissue or portion of an organ may be further combined with a biodegradable polymer. In one embodiment, the system may be supplied with a pusher or plunger to push the molded, irradiated decellularized tissue or portion of an organ into the tract. In one embodiment, the system may be supplied with medical-grade string or suture to pull the molded, irradiated decellularized tissue or portion of an organ into or out of the tract. In one embodiment, the system may be used to deploy the molded, irradiated decellularized tissue or portion of an organ into anal fistulas, vaginal fistulas, biliary fistulas, gastrointestinal fistulas, bladder fistulas and esophageal fistulas.

### Exemplary Sources of Organs and Tissues for Decellularized Extracellular Matrix (ECM)

A tissue is a group of cells with a common structure and function, e.g., epithelial tissue, connective tissue, muscle tissue (skeletal, cardiac, or smooth muscle), and nervous tissue, and includes a pliable sheet that covers or lines or connects organs. An organ is a collection of tissues (two or more) joined in structural unit to serve a common function. Organs include but are not limited to the brain, liver, pancreas, bone, spleen, heart, stomach, kidney, lungs, whole muscles, thymus, anus, and intestine. As used herein, an organ can include perfusable whole organs, or parts of an organ, or vascularized structures thereof, and a tissue includes any structures that contain vascularized tissues, e.g., a trachea.

The ECM of an organ or tissue, or a vascularized portion thereof, may be obtained from any source including, without limitation, heart, liver, lungs, skeletal muscles, brain, pancreas, spleen, kidneys, uterus, eye, spinal cord, intestine, omentum, whole muscle, or bladder, or any portion thereof (e.g., an aortic valve, a mitral valve, a pulmonary valve, a tricuspid valve, a pulmonary vein, a pulmonary artery, coronary vasculature, septum, a right atrium, a left atrium, a right ventricle, or a left ventricle). A solid organ refers to an organ that has a “substantially closed” vasculature system. A “substantially closed” vasculature system with respect to an organ means that, upon perfusion with a liquid, the majority of the liquid can be contained within the solid organ or pass out the native vascular structures and does not leak out of the solid organ, assuming the major vessels are cannulated, ligated, or otherwise restricted. Despite having a “substantially closed” vasculature system, many of the organs listed above have defined “entrance” and “exit” vessels which can be useful for introducing and moving the liquid throughout the organ during perfusion. In addition, other types of vascularized organs or tissues such as, for example, all or portions of joints (e.g., knees, shoulders, or hips), anus, trachea, or spinal cord, can be perfusion decellularized. Further, avascular tissues such as, for example, cartilage or cornea, may be decellularized when part of a larger vascularized structures such as a whole leg.

### Decellularization of Organs or Tissues

Decellularization generally includes the following steps: stabilization of the solid organ, e.g., a vascularized structure thereof, or tissue, decellularization of the solid organ or tissue, renaturation and/or neutralization of the solid organ or tissue, washing the solid organ, degradation of any DNA remaining on the organ, disinfection of the organ or tissue and homeostasis of the organ.

The initial step in decellularizing an organ vascularized structure or tissue can include to cannulate the organ or tissue. The vessels, ducts, and/or cavities of an organ or tissue may be cannulated using methods and materials known in the art. Next, the cannulated organ vascuarlized structure or tissue can be perfused with a cellular disruption medium. Perfusion through an organ can be multi-directional (e.g., antegrade and retrograde). Alternatively, decellularization can be achieved by immersion of an organ or portion thereof into a cellular disruption medium. Methods described herein can include the use of perfusion or immersion decellularized extracellular matrices.

Langendorff perfusion of a heart is routine in the art, as is physiological perfusion (also known as four chamber working mode perfusion). Sec, for example, Dehnert, The Isolated Perfused Warm-Blooded Heart According to Langendorff, In Methods in Experimental Physiology and Pharmacology: Biological Measurement Techniques V. Biomesstechnik-Verlag March GmbH, West Germany, 1988.

Briefly, for Langendorff perfusion, the aorta can be cannulated and attached to a reservoir containing physiological solution to allow the heart to function outside of the body for a specified duration of time. To achieve perfusion decellularization the protocol has been modified to perfuse a cellular disruption medium delivered in a retrograde direction down the aorta either at a constant flow rate delivered, for example, by an infusion or roller pump or by a constant hydrostatic pressure pump. In both instances, the aortic valves can be forced shut and the perfusion fluid can be directed into the coronary ostia (thereby perfusing, via antegrade, the entire ventricular mass of the heart), which then drains into the right atrium via the coronary sinus. For working mode perfusion, a second cannula can be connected to the left atrium and perfusion can be changed to retrograde.

In one embodiment, a physiological solution can include phosphate buffer saline (PBS). In one embodiment, the physiological solution can be a physiologically compatible buffer supplemented with, e.g., nutritional supplements (for instance, glucose). For example, for heart, the physiological solution may be Modified Krebs-Henseleit buffer having 118 mM NaCl, 4.7 mM KCl, 1.2 mM MgSO₄, 1.2 mM KH₂PO₄, 25 mM NaHCO₃, 11 mM glucose, 1.75 mM CaCl₂), 2.0 mM pyruvate and 5 U/L insulin; or Krebs buffer containing 118 mM NaCl, 4.7 mM KCl, 25 mM NaHCO₃, 1.2 mM MgSO₄, 1.2 mM KH₂PO₄, 2 mM CaCl₂) gassed with 95% O₂, 5% CO₂. Hearts may be perfused with glucose (e.g., about 11 mM) as a sole substrate or in combination with about 1 or 1.2 mM palmitate. For kidney, the physiological solution may be KPS-1® Kidney Perfusion Solution. For liver, the physiological solution may be Krebs-Henseleit buffer having 118 mM NaCl, 4.7 mM KCl, 1.2 mM MgSO₄, 1.2 mM KH₂PO₄, 26 mM NaHCO₃, 8 mM glucose, and 1.25 mM CaCl₂) supplemented with 2% BSA.

One or more cellular disruption media may be used to decellularize an organ or tissue. A cellular disruption medium generally includes at least one detergent such as but not limited to SDS, PEG, CHAPS or Triton X. A cellular disruption medium can include water such that the medium can be osmotically incompatible with the cells. Alternatively, a cellular disruption medium can include a buffer (e.g., PBS) for osmotic compatibility with the cells. Cellular disruption media also may include enzymes such as, without limitation, one or more collagenases, one or more dispases, one or more DNases, or a protease such as trypsin. In some instances, cellular disruption media also or alternatively may include inhibitors of one or more enzymes (e.g., protease inhibitors, nuclease inhibitors, and/or collegenase inhibitors).

In certain embodiments, a cannulated organ or tissue may be perfused sequentially with two different cellular disruption media. For example, the first cellular disruption medium may include an anionic detergent such as SDS and the second cellular disruption medium can include an ionic detergent such as Triton X. Following perfusion with at least one cellular disruption medium, a cannulated organ or tissue may be perfused, for example, with wash solutions and/or solutions containing one or more enzymes such as those disclosed herein.

Alternating the direction of perfusion (e.g., antegrade and retrograde) may assist in decellularizing the entire organ or tissue. Decellularization generally decellularizes the organ from the inside out, resulting in very little damage to the ECM. An organ or tissue may be decellularized at a suitable temperature between 4 and 40° C. Depending upon the size and weight of an organ or tissue and the particular detergent(s) and concentration of detergent(s) in the cellular disruption medium, an organ or tissue generally can be perfused from about 0.05 hours to about 5 hours, per gram of solid organ or tissue (generally >50 grams), or about 2 hours to about 12 hours, per gram of solid organ or tissue for organs (generally <50 grams), with cellular disruption medium. Including washes, an organ may be perfused for up to about 0.75 hours to about 10 hours per gram of solid organ or tissue (generally >50 grams), or about 12 hours to about 72 hours, per gram of tissue (generally <50 grams). Decellularization time can be dependent upon the vascular and cellular density of the organ or tissue with limited scaling for overall mass. Therefore, as general guidance the time ranges and masses above are provided. Perfusion generally can be adjusted to physiologic conditions including pulsatile flow, rate and pressure.

A decellularized organ or tissue has the extracellular matrix (ECM) component of all or most regions of the organ or tissue, including ECM components of the vascular tree. ECM components can include any or all of the following: fibronectin, fibrillin, laminin, elastin, members of the collagen family (e.g., collagen I, III, and IV), ECM associated growth proteins including growth factors and cytokines, glycosaminoglycans, ground substance, reticular fibers and thrombospondin, which can remain organized as defined structures such as the basal lamina. Successful decellularization can be defined as the absence of detectable myofilaments, endothelial cells, smooth muscle cells, and nuclei in histologic sections using standard histological staining procedures or removal of over 97% of detectable DNA as measured by fluorometric assay. Residual cell debris may be removed from the decellularized organ or tissue.

The morphology and the architecture of the ECM can be maintained during and following the process of decellularization. “Morphology” as used herein refers to the overall shape of the organ, tissue or of the ECM, while “architecture” as used herein refers to the exterior surface, the interior surface, and the ECM therebetween.

The morphology and architecture of the ECM may be examined visually and/or histologically. For example, the basal lamina on the exterior surface of a solid organ or within the vasculature of an organ or tissue should not be removed or significantly damaged due to perfusion decellularization. In addition, the fibrils of the ECM should be similar to or significantly unchanged from that of an organ or tissue that has not been decellularized.

One or more compounds may be applied in or on a decellularized organ or tissue to, for example, preserve the decellularized organ, or to prepare the decellularized organ or tissue for recellularization and/or to assist or stimulate cells during the recellularization process. Such compounds include, but are not limited to, one or more growth factors (e.g., VEGF, DKK-1, FGF, BMP-1, BMP-4, SDF-1, IGF, and HGF), immune modulating agents (e.g., cytokines, glucocorticoids, IL2R antagonist, leucotriene antagonists), and/or factors that modify the coagulation cascade (e.g., aspirin, heparin-binding proteins, and heparin). In addition, a decellularized organ or tissue may be further treated with, for example, irradiation (e.g., UV, gamma) to reduce or eliminate the presence of any type of microorganism remaining on or in a decellularized organ or tissue.

### Recellularization of Organs or Tissues

A molded, irradiated decellularized portion of an organ or tissue may be contacted with a population of cells, either differentiated (mature or primary) cells, stem cells, or partially differentiated cells. Thus, the cells can be totipotent cells, pluripotent cells, or multipotent cells, and can be uncommitted or committed, and may be single-lineage cells. The cells may be undifferentiated cells, partially differentiated cells, or fully differentiated cells including fetal derived cells. Cells may include progenitor cells, precursor cells, or “adult” derived stem cells including umbilical cord cells and fetal stem cells. Cells useful in the matrices of the invention include embryonic stem cells (as defined by the National Institute of Health (NIH); see, for example, the Glossary at stemcells.nih.gov on the World Wide Web) and iPS cells.

Examples of cells that can be used to recellularize a portion of an organ or tissue include, without limitation, embryonic stem cells, umbilical cord blood cells, tissue-derived stem or progenitor cells, bone marrow-derived step or progenitor cells, blood-derived stem or progenitor cells, mesenchymal stem cells (MSC), skeletal muscle-derived cells, multipotent adult progentitor cells (MAPC), or iPS cells Additional cells that can be used include cardiac stem cells (CSC), multipotent adult cardiac-derived stem cells, cardiac fibroblasts, cardiac microvasculature endothelial cells, aortic endothelial cells, coronary endothelial cells, microvascular endothelial cells, venous endothelial cells, arterial endothelial cells, smooth muscle cells, cardiomyocytes, hepatocytes, beta-cells, keratinocytes, purkinji fibers, neurons, bile duct epithelial call, islet cells, pneumocytes, clara cells, brush boarder cells, or podocytes. Bone marrow-derived stem cells such as bone marrow mononuclear cells (BM-MNC), endothelial or vascular stem or progenitor cells, and peripheral blood-derived stem cells such as endothelial progenitor cells (EPC) may also be used as cells.

The number of cells that are introduced into and onto a perfusion decellularized scaffold may depend both the organ (e.g., which organ, the size and weight of the organ) or tissue and the type and developmental stage of the regenerative cells. Different types of cells may have different tendencies as to the population density those cells will reach. Similarly, different organ or tissues may be cellularized at different densities. By way of example, a decellularized organ or tissue can be “seeded” with at least about 500, 1,000 (e.g., at least 10,000, 100,000, 1,000,000, 10,000,000, or 100,000,000) cells; or can have from about 1,000 cells/mg tissue (wet weight, e.g., prior to decellularization) to about 10,000,000 cells/mg tissue (wet weight) attached thereto.

Cells can be introduced (“seeded”) into a decellularized organ or tissue by injection into one or more locations. In addition, more than one type of cell may be introduced into a decellularized organ or tissue. For example, a population of differentiated cell types can be injected at multiple positions in a decellularized organ or tissue or different cell types may be injected into different portions of a decellularized organ or tissue. Alternatively, or in addition to injection, cells or a cocktail of cells may be introduced by perfusion into a cannulated decellularized organ or tissue. For example, cells can be perfused into a decellularized organ using a perfusion medium, which can then be changed to an expansion and/or differentiation medium to induce growth and/or differentiation of the cells. Location specific differentiation may be achieved by placing cells into the various locations within the organ, e.g., into regions of the heart, such as, atrial, ventricular or nodal.

During recellularization, a portion of an organ or tissue can be maintained under conditions in which at least some of the cells can multiply and/or differentiate within and on the decellularized organ or tissue. Those conditions include, without limitation, the appropriate temperature and/or pressure, electrical and/or mechanical activity, force, the appropriate amounts of O₂ and/or CO₂, an appropriate amount of humidity, and sterile or near-sterile conditions. During recellularization, the decellularized portion of an organ or tissue and the regenerative cells attached thereto can be maintained in a suitable environment. For example, the cells may require a nutritional supplement (e.g., nutrients and/or a carbon source such as glucose), exogenous hormones or growth factors, and/or a particular pH.

Cells may be allogeneic to a decellularized organ or tissue (e.g., a human decellularized organ or tissue seeded with human cells), or cells may be xenogeneic to a decellularized organ or tissue (e.g., a pig decellularized organ or tissue seeded with human cells). “Allogeneic” as used herein refers to cells obtained from the same species as that from which the organ or tissue originated (e.g., related or unrelated individuals), while “xenogeneic” as used herein refers to cells obtained from a species different than that from which the organ or tissue originated.

Perfusion decellularized matrices of organs with a substantially closed vascular system can be useful because perfusion decellularization preserves the intact matrix and microenvironment, including an intact vascular and microvascular system, that vascular system, or ducts or other conduits, may be utilized to deliver cells as well as nutrients and/or differentiation or maintenance factors, to the cells in vitro. Cells and nutrients and/or other factors may be delivered by other means, e.g., injection, or passive means, or a combination thereof. In one embodiment, a cell population of interest can be perfused into the perfusion decellularized organ ECM after inflation allowing for the seeding into the interstitial space or matrix outside of the vascular conduits. This includes the active migration and/or homing of cells to their native microstructure, e.g. the homing of endothelial cells to the vasculature. In one embodiment, a cell population of interest can be perfused into the perfusion decellularized ECM followed by a second cell population, e.g., a beta cell population can be introduced followed by an endothelial cell population, where the endothelial cells remain in the vascular conduits as in their native microenvironment. In one embodiment, a cell population of interest can be perfused into the perfusion decellularized ECM after inflation followed by a second cell population, e.g., an endothelial cell population can be introduced followed by a population of cells that include beta cells, where the endothelial cells remain in the vascular conduits as in their native microenvironment. In another embodiment, two or more cell populations can be combined and perfused together. In another embodiment, two or more distinct cell populations can be introduced serially through either perfusion, direct injection or a combination of both.

The cells may be introduced in media that support the proliferation, metabolism, and/or differentiation of the cells. Alternatively, after the cells have populated the ECM, the medium can be changed to one that supports the proliferation, metabolism and differentiation of the cells. The cultured cells may exist in the ECM at physiological cell densities and, in the presence of media that support the proliferation, metabolism, and/or differentiation of the cells and/or the appropriate microenvironment in the ECM, allow for the maintenance and/or functional differentiation of the cells.

Stem or progenitor media may contain a variety of components including, for example, KODMEM medium (Knockout Dulbecco's Modified Eagle's Medium), DMEM, Ham's F12 medium, FBS (fetal bovine serum), FGF2 (fibroblast growth factor 2), KSR or hLIF (human leukemia inhibitory factor). The cell differentiation media may also contain supplements such as L-Glutamine, NEAA (non-essential amino acids), P/S (penicillin/streptomycin), N2, B27 and beta-mercaptocthanol. It is contemplated that additional factors may be added to the cell differentiation media, including, but not limited to, fibronectin, laminin, heparin, heparin sulfate, retinoic acid, members of the epidermal growth factor family (EGFs), members of the fibroblast growth factor family (FGFs) including FGF2, FGF7, FGF8, and/or FGF10, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, high dose activin, and amnionless or variants or functional fragments thereof. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage, or variants or functional fragments thereof. Other growth factors may include members of the insulin like growth factor family (IGF), insulin, the wingless related (WNT) factor family, and the hedgehog factor family or variants or functional fragments thereof. Additional factors may be added to promote mesendoderm stem/progenitor, endoderm stem/progenitor, mesoderm stem/progenitor, or definitive endoderm stem/progenitor proliferation and survival as well as survival and differentiation of derivatives of these progenitors.

In one embodiment, perfusion decellularized matrices can be combined with iPS or ES cells differentiated using the embryoid body (EB) method. For example, human iPS cell lines reprogrammed by transduction, e.g., lentiviral-mediated transduction, of transcription factors (OCT4, SOX2, NANOG and LIN28; Oct3/4, Sox2, Klf4, and c-Myc; or Oct3/4, Sox2, and Klf4) can be employed. iPS clones of fetal origin or of newborn origin may be employed. Human ES cell lines may also be employed. iPS cells and ES cells may be maintained on irradiated mouse embryonic fibroblasts (MEFs) at a density of 19,500 cells/cm² in 6-well culture plates (Nunc) in DMEM/F12 culture medium supplemented with 20% KnockOut serum replacer (Invitrogen), 0.1 mmol/L nonessential amino acids, 1 mmol/L L-glutamine, and 0.1 mmol/L β-mercaptocthanol (Sigma). In addition, the medium may be supplemented with 100 ng/mL, zebrafish basic fibroblast growth factor for iPS cells, and with 4 ng/mL human recombinant basic fibroblast growth factor (Invitrogen) for hES cells. iPS and ES cell lines may also be maintained on gelatinized 100-mm dishes in DMEM (Sigma-Aldrich) containing 15% fetal calf serum (FCS; Sigma-Aldrich), 0.1 μmol/L 2-mercaptoethanol (2ME), and 1,000 units/ml LIF (Chemicon International). For differentiation, these cells may treated with 0.25% Trypsin/ethylenediaminetetraacetic acid (GIBCO), and transferred to gelatinized 6-well plates in α-minimum essential medium (GIBCO) supplemented with 10% FCS and 0.05 μmol/L 2ME, at a concentration of 3×10⁴ cells/well.

Colonies may be detached from culture plates by incubating with 1 mg/mL dispase (Gibco) solution at 37° C. for 8 to 15 minutes and placed in ultralow attachment plates in suspension culture, e.g., for 4 days. During suspension culture, the medium may be changed at day 1 followed by culture for another 3 days without medium change. EBs can then be plated on 0.1% gelatin-coated culture plates, e.g., at the density or 50 to 100 EBs per well, or in the perfusion decellularized ECM and cultured in differentiation medium (e.g., changed daily).

In some instances, an organ or tissue generated by the methods described herein can be transplanted into a patient. In those cases, the cells used to recellularize a decellularized organ or tissue can be obtained from the patient such that the cells are “autologous” to the patient. Cells from a patient can be obtained from, for example, blood, bone marrow, tissues, or organs at different stages of life (e.g., prenatally, neonatally or perinatally, during adolescence, or as an adult) using methods known in the art. Alternatively, cells used to recellularize a decellularized organ or tissue may be syngeneic (i.e., from an identical twin) to the patient, cells can be human lymphocyte antigen (HLA)-matched cells from, for example, a relative of the patient or an HLA-matched individual unrelated to the patient, or cells can be allogeneic to the patient from, for example, a non-HLA-matched donor.

Irrespective of the source of the cells (e.g., autologous or not), the decellularized solid organ can be autologous, allogeneic or xenogencic to a patient.

The progress of cells can be monitored during recellularization. For example, the number of cells on or in an organ or tissue can be evaluated by taking a biopsy at one or more time points during recellularization. In addition, the amount of differentiation that cells have undergone can be monitored by determining whether or not various markers are present in a cell or a population of cells. Markers associated with different cells types and different stages of differentiation for those cell types are known in the art, and can be readily detected using antibodies and standard immunoassays. See, for example, Current Protocols in Immunology, 2005, Coligan et al., Eds., John Wiley & Sons, Chapters 3 and 11. Nucleic acid assays as well as morphological and/or histological evaluation can be used to monitor recellularization.

### Exemplary Uses for the Molded, Irradiated ECM

The ECM may be molded to form a product useful to treat, for example, diseases of the eye, adnexa, car, and mastoid process including but not limited to lacrimal fistula, mastoid fistula craniofistula, e.g., between the intracranial space and a paranasal sinus, labyrinthine fistula, perilymph fistula, or preauricular fistula; diseases of the circulatory system including but not limited to coronary arteriovenous fistula, arteriovenous fistula, e.g., of the pulmonary vessels cerebral arteriovenous fistula, acquired, or fistula of an artery; diseases of the respiratory system including but not limited to pyothorax with fistula or tracheoesophageal fistula; diseases of the digestive system including but not limited to duodeno biliary fistula, e.g., salivary gland fistula, fistula of stomach and duodenum, gastrocolic fistula, gastrojejunocolic fistula, enterocutaneous fistula, gastric fistula from the stomach to the skin surface, fistula of appendix, anal and rectal fissures and fistulas, e.g., anal fistula or anorectal fistula (fecal fistula, fistula-in-ano), fistula of intestine, e.g., enteroenteral fistula: between two parts of the intestine, fistula of gallbladder or fistula of bile duct, e.g., biliary fistula, or pancreatic fistula: between the pancreas and the exterior via the abdominal wall; diseases of the musculoskeletal system and connective tissue including fistula of joint; diseases of the urogenital system including but not limited to vesicointestinal fistula, urethral fistula, fistula of nipple, fistulae involving female genital tract/obstetric fistula including, e.g., vesicovaginal fistula, cervical fistula, enterovaginal fistula: between the intestine and the vagina, rectovaginal fistula, other female intestinal-genital tract fistulae, or female genital tract-skin fistulae; congenital malformations, deformations and chromosomal abnormalities including but not limited to sinus, fistula and cyst of branchial cleft, congenital preauricular fistula, portal vein-hepatic artery fistula, congenital fistula of lip, congenital fistula of salivary gland, congenital absence, atresia and stenosis of rectum with fistula, congenital absence, atresia and stenosis of anus with fistula, congenital fistula of rectum and anus, congenital fistulae between uterus and digestive and urinary tracts, or congenital rectovaginal fistula; or external causes including but not limited to traumatic arteriovenous fistula or persistent postoperative fistula.

For example, a hydrated plug (with or without a delivery device) can be inserted in an internal opening and then pulled through the tract until light resistance can be met and then sutured securely in the primary opening. Excess plug from the external opening may be trimmed at the skin level. The tract may be irrigated with hydrogen peroxide or other anti-microbial solution before insertion. The external opening may be partially open as this is the path that allows drainage and prevents a closed-space infection. The maturing of the tract, making the wall more fibrotic, which results in increased healing, may minimize sepsis and facilitate fistula closure when used in conjunction with other procedures, such as an advancement flap.

Nerve scarring can cause severe pain and dysfunction. Treatment of the scarred nerve frequently yields unpredictable results. A barrier wrap around the scarred nerve may be of benefit in preventing the recurrence of epineural scarring following neurolysis. Nerve wraps may inhibit recurrent nerve tissue adhesions and diminish inflammatory and immunologic reactions in peripheral nerve surgery. Nerve wraps may serve as a nerve guide, e.g., across nerve discontinuities, and a nerve protector, e.g., following decompression procedures or nerve isolation in traumatized nerve beds or closure around partially severed nerves. Exemplary lengths and diameters of nerve wraps include a length of about 2.5 cm or 5.0 cm and a diameter of about 4 mm, 6 mm, or 12 mm).

Tendon wraps can be an interface between the tendon and the tendon sheath or the surrounding tissue and may be used in the treatment of bowed tendons, occelets, splints, shins and suspensory injuries, tendon repair surgery, including reinforcement of the rotator cuff, patella, achilles, biceps, quadriceps, or other tendons.

While exemplary embodiments have been shown and described herein, it will be obvious to those skilled in the art that such embodiments are provided by way of example only. Numerous variations, changes, and substitutions will occur to those skilled in the art. It should be understood that various alternatives to the embodiments described herein may be employed. It is intended that the following claims define the scope of the disclosure and that methods and structures within the scope of these claims and their equivalents be covered thereby.

1-52. (canceled)

53. A method of shape formation, comprising:
providing a decellularized and dehydrated extracellular matrix (ECM) from a mammalian organ or tissue;
placing a portion of the decellularized and dehydrated ECM into a mold; and
compressing the portion of the decellularized and dehydrated ECM within the mold so that it retains the shape of the mold,
wherein the decellularized and dehydrated ECM maintains a scaffold of collagen fibers surrounding empty cellular compartments that existed before decellularization and dehydration.

54. The method of claim 53, wherein the shaped portion of the decellularized and dehydrated ECM does not comprise a surgical mesh.

55. The method of claim 53, further comprising irradiating the decellularized and dehydrated ECM within the mold with a dose of radiation.

56. The method of claim 55, wherein irradiating the decellularized and dehydrated ECM within the mold comprises applying E-beam or gamma radiation to the decellularized and dehydrated ECM.

57. The method of claim 55, wherein the dose of radiation is between about 5 kGy to about 50 kGy.

58. The method of claim 53, wherein the provided decellularized and dehydrated ECM has been inflated with a gas or vapor.

59. The method of claim 53, further comprising placing the shaped portion of the decellularized and dehydrated ECM into a deployment device.

60. The method of claim 59, wherein the deployment device comprises a hollow tube and a plunger or pusher designed to expel the shaped portion of the decellularized and dehydrated ECM into a body tract.

61. The method of claim 53, further comprising compressing the shaped portion of the decellularized and dehydrated ECM onto an elongate member.

62. The method of claim 61, wherein the elongate member is a medical-grade string or suture.

63. The method of claim 53, wherein the dimensions of the portion of the decellularized and dehydrated ECM placed into the mold range from about 0.5 cm×0.5 cm×0.5 cm to about 30 cm×30 cm×30 cm.

64. The method of claim 53, wherein the dimensions of the shaped portion of the decellularized and dehydrated ECM comprise a diameter and a length, and wherein the length is at least two times greater than the diameter.

65. The method of claim 64, wherein the length is at least three times greater than the diameter.

66. The method of claim 53, wherein the mammalian organ or tissue is selected from the group consisting of liver, lung, kidney, muscle, spleen, and heart.

67. The method of claim 53, wherein the mammalian organ or tissue is obtained from a pig.

68. The method of claim 53, wherein placing the portion of the decellularized and dehydrated ECM into the mold comprises placing multiple layers of decellularized and dehydrated ECM into the mold.

69. A method, comprising:
placing a shaped portion of decellularized and dehydrated ECM into a wound site of a mammal, wherein the shaped portion of decellularized and dehydrated ECM is formed by:
providing a decellularized and dehydrated ECM from a mammalian organ or tissue;
placing a portion of the decellularized and dehydrated ECM into a mold; and
compressing the portion of the decellularized and dehydrated ECM within the mold so that it retains the shape of the mold,
wherein the decellularized and dehydrated ECM maintains a scaffold of collagen fibers surrounding empty cellular compartments that existed before decellularization and dehydration.

70. The method of claim 69, wherein placing the shaped portion of decellularized and dehydrated ECM into the wound site comprises using a plunger or pusher to expel the shaped portion from a hollow tube of a deployment device.

71. The method of claim 69, wherein placing the shaped portion of decellularized and dehydrated ECM into the wound site comprises pulling a medical-grade string or suture.

72. The method of claim 71, further comprising suturing the shaped portion of decellularized and dehydrated ECM in place within the wound site.

73. A system, comprising:
a shaped portion of decellularized and dehydrated ECM formed by compressing the ECM in a mold to retain the mold's shape,
wherein dimensions of the shaped portion of the ECM comprise a diameter and a length, with the length being at least two times greater than the diameter, and
wherein the ECM maintains a scaffold of collagen fibers surrounding empty cellular compartments that existed before decellularization and dehydration.

74. The system of claim 73, further comprising a deployment device comprising a hollow tube and a plunger or pusher designed to expel the shaped portion of the decellularized and dehydrated ECM into a body tract.

75. The system of claim 73, further comprising an elongate member in the form of a medical-grade string or suture, wherein the shaped ECM is compressed onto the elongate member.
